(19) United States
(12) Patent Application Publication
(10) Pub. No.: US 2009/0036665 A1
(43) Pub. Date: Feb. 5, 2009

(54) Disposable Sample Processing Unit

(76) Inventors: Gonzalo Domingo, Seattle, WA (US); Bernhard Weigl, Seattle, WA (US); Paul Labarre, Seattle, WA (US); Jay Gerlach, Seattle, WA (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 12/172,086

(22) Filed: Jul. 11, 2008

Related U.S. Application Data
(60) Provisional application No. 60/949,199, filed on Jul. 11, 2007.

(51) Int. Cl. C07H 21/00; B01L 11/00

(52) U.S. Cl. 536/25.41; 422/101

(57) ABSTRACT

A low-cost, non-instrumented, easy-to-use disposable platform for extraction, stabilization, and preservation of viral RNA in specimens at the point of collection is described. The system may use chemical heating. The platform performs the following steps: specimen lysis, RNA extraction, and RNA stabilization in a modular approach. This modular approach confers versatility to the product for application to multiple targets such as avian flu, and HIV, specimens such as blood, nasal swabs, and downstream applications such as PCR or transcription-mediated amplification. The technology described is a point-of-care specimen-processing platform generically applicable to both emerging point-of-care and central-facility molecular diagnostic tests, as well as to surveillance applications.

### CROSS REFERENCE TO RELATED APPLICATIONS

This application claims the benefit of U.S. Application No. 60/949,199, filed on Jul. 11, 2007, which is hereby incorporated by reference in its entirety.

### BACKGROUND OF THE INVENTION

1. Field of the Invention

The present invention generally relates to a device for the processing of clinical specimens for molecular diagnostic applications.

2. Background Art

RNA biomarkers are the target analytes for several infectious diseases of high global health importance including HIV, pandemic influenza, and dengue. A major challenge in developing diagnostic tests for RNA-based analytes is specimen handling that protects the integrity of these labile molecules. There are several commercially available products that address this problem. Most of these products are expensive, technically demanding, and/or require some form of refrigeration. These requirements cannot be easily met in low-resource or remote settings as is the case in the majority of the developing world. There is a need for a low-cost, non-instrumented, and simple-to-use clinical specimen processing protocol or device that stabilizes the RNA analyte at the point of care (POC).

Unlike DNA, RNA is labile to hydrolysis and susceptible to prevalent RNAses. As a consequence, although DNA in clinical samples can be stabilized by spotting the DNA on filter paper and allowing it to dry at room temperature, RNA stabilization requires the use of stabilizing agents and refrigeration and/or freezing. The current stabilization protocols such as RNALater from Ambion require use of refrigeration for long-term preservation of specimens and sample transfer from, for example, a clinical site to a clinical laboratory or a surveillance site. The steps required to stabilize RNA in clinical samples are cumbersome and prohibitive for remote clinical settings. Many clinical settings in resource-limited countries do not have reliable refrigeration capacity or technical resources to be able to manage clinical samples. Additionally, stabilizing RNA samples in rural and remote clinical settings is essential for surveillance efforts for potential pandemics such as avian flu. Dried blood spots and dried plasma spots have successfully been used to stabilize HIV-1 RNA for long periods of time at room temperature, however, the sensitivity of assays performed on these specimens drops significantly below a viral load copy number of 4,000 copies/ml. Additionally for some assay formats extraction of RNA from the filter paper is not trivial.

One way to stabilize RNA from clinical samples would be to prepare the corresponding cDNA within minutes of sample collection at the clinical site. In accelerated stability studies, we found cDNA molecules to be more stable than RNA molecules in low-ionic-strength aqueous solution. DNA molecules are also the target analyte for several downstream molecular diagnostic tests. Several kits have been developed to extract RNA from clinical samples but require pipetting of several reagents and centrifugation or a vacuum manifold. Additionally the reverse transcription step requires a heating step for the reverse transcriptase. Again these protocols are too technological and time demanding for typical POC settings, let alone remote clinical settings.

### BRIEF SUMMARY OF THE INVENTION

In one embodiment, a method for processing a clinical sample at a point-of-care site comprises the steps of collecting a clinical sample from a patient at a point-of-care site, extracting nucleic acid from the clinical sample at the point-of-care site, and preserving the extracted nucleic acid at the point-of-care site. The steps of extracting and preserving the nucleic acid occur in a sample processing unit.

In another embodiment, a sample processing kit comprises reagents, a first syringe for holding a clinical sample in a chaotropic lysis reagent, second and third syringes for holding first and second buffers for washing nucleic acid (which may be bound to a silica/glass membrane), a fourth syringe for holding drying air, a fifth syringe for holding air for fluidly motivating an eluent, and a vial containing a reagent comprising chemicals or biochemical agents for conversion of RNA to complimentary DNA, and a disposable device. The disposable device comprises ports adapted to fluidly engage all syringes and the vial, a fluidic network, a nucleic acid capture matrix for extraction of either DNA or RNA or both from the clinical sample, and a chemical heating element. The kit may further comprise reagents which may include target specific primers, random hexamers, and a reverse-transcription mixture. The disposable device comprises the necessary fluidic channels and nucleic acid capture matrix to perform extraction of either DNA or RNA or both from clinical specimens such as whole blood, plasma, sera, blood cells, nasal swabs, nasal washes, urine, stool, or buccal washes. The POC sample processing platform will perform RNA and or DNA extraction and provide stabilized RNA and or DNA in the forms of either clean RNA and or DNA with or without a stabilizing agent, or cDNA or both or a combination of different types of cDNA. Different types of cDNA can be generated with either one or more target-specific DNA primers or random DNA primers, or mRNA DNA primers or any form of DNA primers or combination of these.

In another embodiment, a disposable device comprises a sample port, a fluidic network, a nucleic acid capture matrix for extraction of either DNA or RNA or both from a clinical sample, a chemical heating element, a selectively movable filter operatively connected to the fluidic network, allowing manual switching of the filter from one fluid channel to another fluid channel of the fluidic network, and a collection port.

With such methods and platforms, it is possible to perform DNA and or RNA extraction from clinical specimens at the point-of sampling without the use of additional instrumentation.

### BRIEF DESCRIPTION OF THE DRAWINGS/FIGURES

Features, aspects and advantages of the present invention will become better understood with reference to the following description, appended claims, and accompanying drawings, which are not drawn to scale.

FIG. 1 is a forward perspective view of a disposable sample processing unit.

FIG. 2 is a forward perspective view of the device housing of the disposable sample processing unit of FIG. 1.

FIG. 3 is a perspective view of the filter dial assembly of the disposable sample processing unit of FIG. 1.

FIG. 4 is a reverse perspective view of the disposable sample processing unit of FIG. 1 having a heating pouch assembly disposed in the device housing.

FIG. 5 is a forward perspective view of the filter dial assembly of FIG. 3 disposed in the device housing.

FIG. 6 shows a flowchart of a process for using the disposable sample processing unit to extract and stabilize viral RNA in specimens at the point of collection.

FIGS. 7 A-C are temperature profiles of a heat mixture in a reverse transcription (RT) reaction.

FIGS. 8 A-C are temperature profiles of an RT mixture in a reverse transcription reaction.

FIG. 9 is a graph comparing Q-PCR values for RT performed in the same temperature profiles as those generated by exothermic heat packs.

### DETAILED DESCRIPTION OF THE INVENTION

The present invention is now described with reference to the figures where like reference numbers indicate identical or functionally similar elements. While specific configurations and arrangements are discussed, it should be understood that this is done for illustrative purposes only. A person skilled in the relevant art will recognize that other configurations and arrangements can be used without departing from the spirit and scope of the invention.

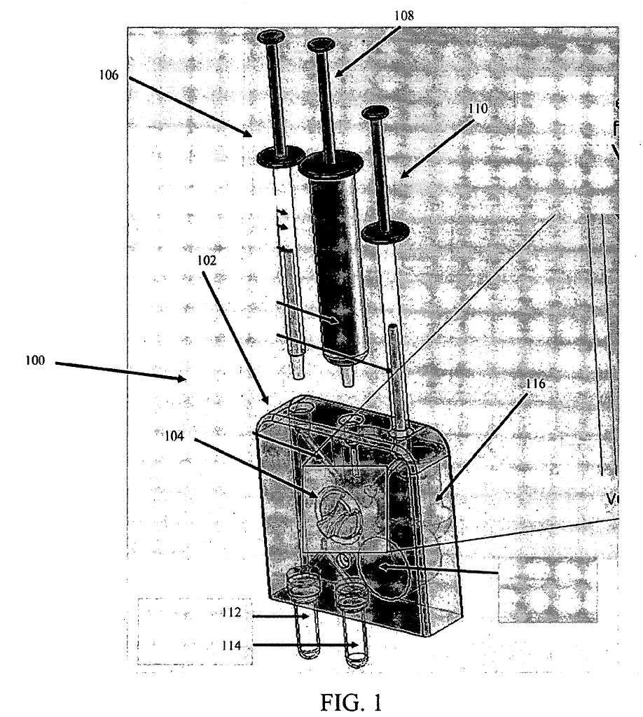

An exemplary disposable sample processing unit 100 is shown in FIG. 1. Disposable sample processing unit 100 may include device housing 102, filter dial assembly 104, heating pouch assembly 116, Point-of-Care (POC) vial 112, and Clinical Facility (CF) vial 114.

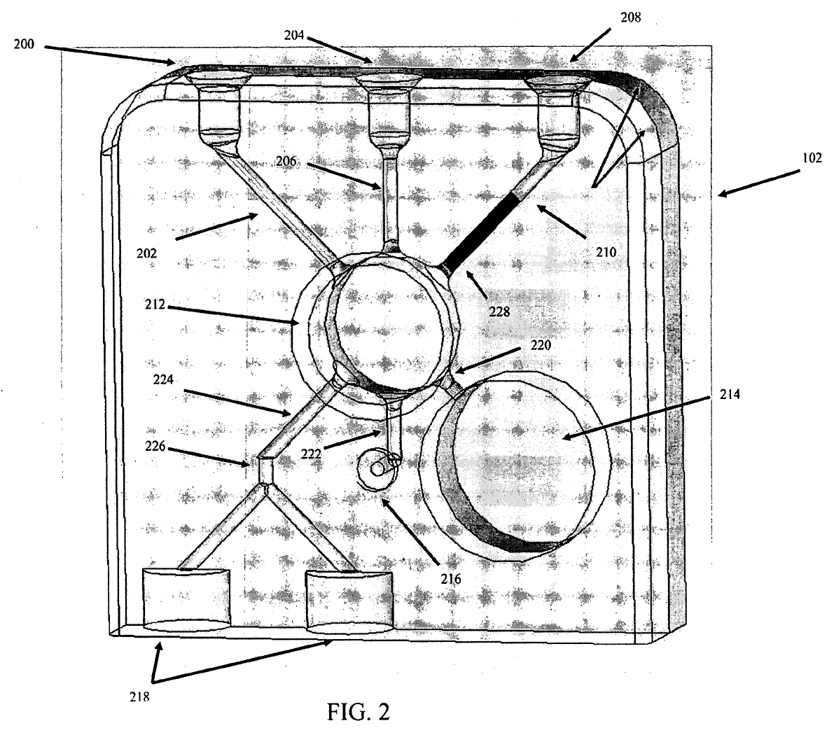

Device housing 102 is but one component of disposable sample processing unit 100 and is shown in greater detail in FIG. 2. Device housing 102 may be made of any material that is appropriate for manufacturing a low-cost, rigid housing. Specifically, housing 102 may be machined from polymeric materials, such as polycarbonate or polypropylene, or it can be machined by injection molding. Assembly of the component halves can be accomplished via thermal welding, ultrasonic welding, or bonding with a pressure sensitive adhesive cut on a laser and precisely positioned between the component halves.

There are several approaches for isolating RNA from specimens. The most simple RNA extraction protocols utilize chaotropic agents to lyse cells and release NA in a RNAse-free (denatured RNAse) medium. The addition of phenol chloroform to guanidinium thiocynate allows fractionation of RNA from protein material and DNA in a single step, but still requires a final RNA precipitation step. To avoid centrifugation, sample processing unit 100 is designed to use the approach commonly used in commercial kits of specimen denaturation with the chaotropic agent guanidinium thiocyanate, followed by binding to a silica membrane. This chemistry has been shown to be successful in the extraction of HIV-1 RNA.

Device housing 102 includes intake ports that receive samples for testing, buffer washes, and drying air. Sample and wash port 200, drying air port 204, and eluent-channel air port 208 are disposed on the top face of housing 102 and are sized to accommodate syringe needles. Adapters may be placed in the intake ports to accommodate variations in syringe diameters. Each of these intake ports is separately connected to filter cavity 212. Sample and wash port 200 is fluidly connected to filter cavity 212 by means of sample intake channel 202. Drying air port 204 is fluidly connected to filter cavity 212 by means of drying air intake channel 206. Eluent-channel air port 208 is fluidly connected to filter cavity 212 by means of eluent intake channel 210.

Device housing 102 also includes various exhaust mechanisms. Vial ports 218 are sized to accommodate Eppendorf vials 112 and 114. Each port also includes threaded mating surfaces to enable users to screw threaded vials directly into device housing 102.

Vent 216 allows and regulates air flow between the interior of the device housing 102 and the atmosphere. Waste reservoir 214 is designed to receive waste exhausted from the filter cavity 212.

Filter cavity 212 is fluidly connected to these exhaust mechanisms. Filter cavity 212 is connected to vial ports 218 by means of eluent exhaust channel 224. In one embodiment, eluent exhaust channel 224 is bifurcated by eluent splitter 226 so as to allow equal portions of eluent to flow into vial ports 218. Eluent splitter 226 evenly divides eluent by means of carefully designed channel geometry, as would be apparent to one of ordinary skill in the art, such as, for example, that used in multi-tip eppendorfs designed to evenly distribute volumes of fluid.

In another embodiment (not shown), equal distribution of eluent would be achieved by pre-filling two vented equal volume wells above the entrances to vials 112 and 114. Such wells are designed such that one overflows into the other before an air plug pushes the split eluents into POC vial 112 and CF vial 114.

In another embodiment (not shown), POC vial 112 and CF vial 114 are connected in series. Overflow from the first vial fills into the second vial ensuring equal distribution of eluent volume. Both vials may be vented with a hydrophobic Porex™ filter to prevent pressure build-up and loss of eluent.

In another embodiment (not shown), a single vial (either a POC vial 112 or a CF vial 114) is connected to the device with the eluent exhaust channel 224 directing fluid from the filter cavity 212 to a single vial port 218. Filter cavity 212 is fluidly connected to vent 216 by means of drying air exhaust channel 222. Filter cavity 212 is fluidly connected to waste reservoir 214 by means of eluent exhaust channel 220.

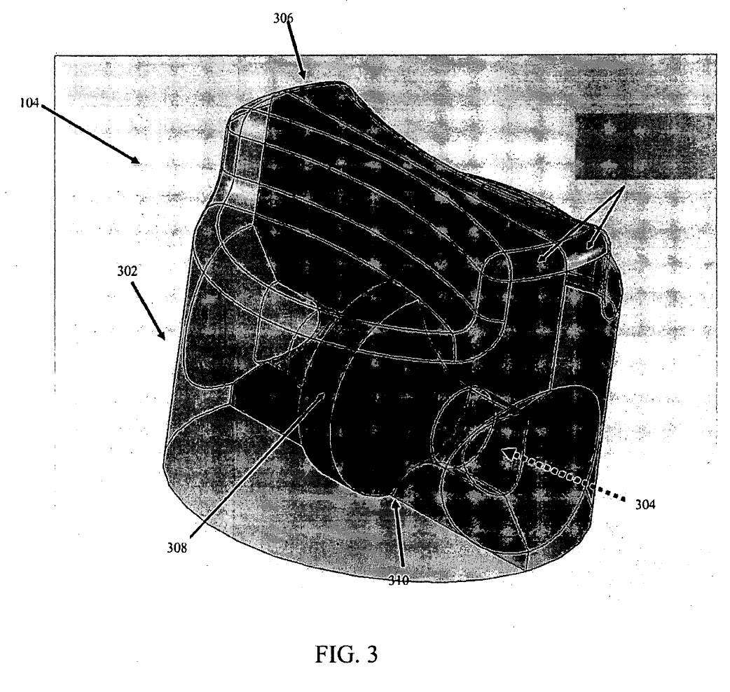

Filter dial assembly 104 is housed within filter cavity 212 and is shown in greater detail in FIG. 3. Filter cavity 212 is designed to allow filter dial assembly 104 to rotate around its center axis enabling filter channel 304 to engage in any one of three positions. While in position 1, filter channel 304 fluidly connects sample port 200 with waste reservoir 214. While in position 2, filter channel 304 fluidly connects drying air port 204 with vent 216. While in position 3, filter channel 304 fluidly connects eluent-channel air port 208 with eluent splitter 226.

Filter dial assembly 104 includes a filter dial 302 and silica bead matrix filter 308. Filter dial 302 may be made of any material that is appropriate for manufacturing a low-cost, rigid dial. Specifically, filter dial 302 may be machined from polymeric materials, such as polycarbonate or polypropylene, or it can be machined by injection molding. Assembly of the component halves can be accomplished via thermal welding, ultrasonic welding, or bonding with a pressure sensitive adhesive cut on a laser and precisely positioned between the component halves. Filter dial 302 includes dial knob 306, which is designed to allow manual rotation of filter dial assembly 104. Dial knob 306 may be aligned with filter channel 304 so as to visually indicate which fluidic channel is engaged.

Silica matrix 308 is disposed in silica matrix cavity 310 within filter dial 302. In one embodiment, silica matrix 308 is a conventional silica bead matrix filter and includes silica beads contained between two porous membranes and a support matrix. In another embodiment, silica matrix 308 may be a conventional glass membrane and a support matrix. In another embodiment, silica matrix 308 may be a combined glass and support matrix material. Silica matrix cavity 310 is orientated within filter dial 302 to allow liquid or air to flow through filter channel 304 and through silica matrix 308.

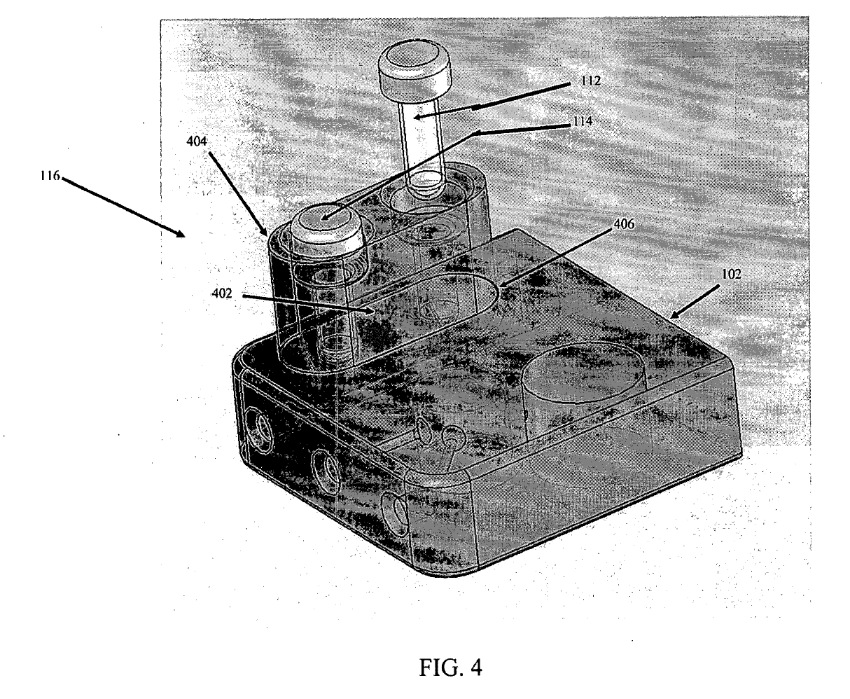

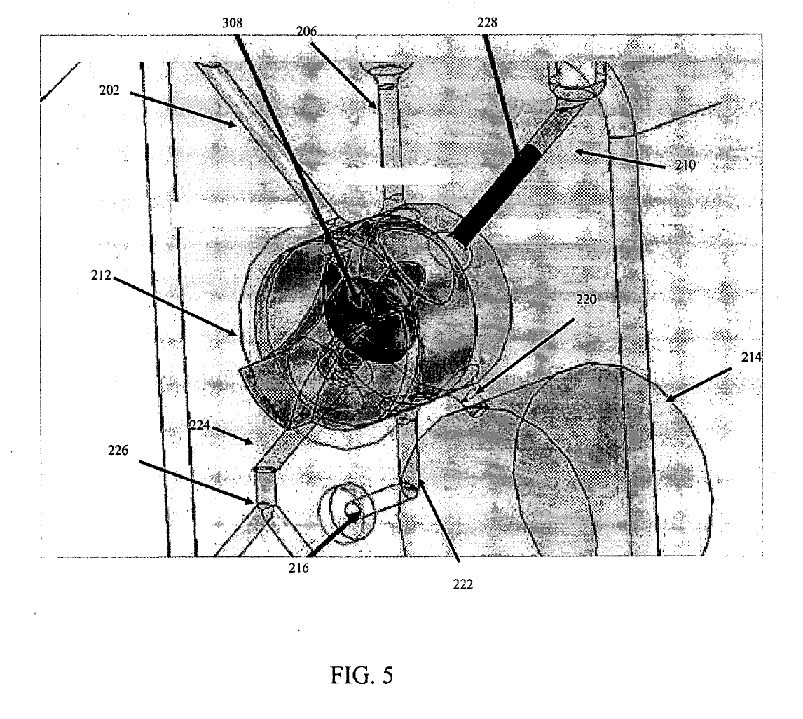

Heating pouch assembly 116 includes heating pouch housing 404 and exothermal chemical device 402 and is shown in more detail in FIG. 4. Heating pouch housing 404 may be made of any material that is appropriate for manufacturing a low-cost, rigid housing. Specifically, heating pouch housing 404 may be machined from polymeric materials, such as polycarbonate or polypropylene, or it can be machined by injection molding. Assembly of the component halves can be accomplished via thermal welding, ultrasonic welding, or bonding with a pressure sensitive adhesive cut on a laser and precisely positioned between the component halves.

Chemical temperature control (heat and/or cooling) is preferred to electrical means such as platinum film resistors or Peltier thermocouples because it does not require external energy sources. Additionally, some chemical reactions are capable of self-regulating temperature thereby eliminating the requirement for RTD temperature detection and proportional-integral-derivative (PID) controls. Chemical heating/cooling elements are particularly well-suited for microfluidic devices because the mass of the reagents can be very small. In one embodiment, exothermal chemical device 402 can be augmented and/or replaced with electrical means. In another embodiment, device 402 could be replaced with an endothermic chemical device. Chemical temperature control is described in greater detail in U.S. patent application Ser. No. 12/134,965, filed Jun. 6, 2008, entitled “Chemical Temperature Control,” the disclosure of which is hereby incorporated by reference in its entirety.

In order to minimize heat losses, insulation (not shown) may be used. Urethane foam, besides being an excellent insulator, is also cheap and easy to incorporate into various devices. Alternately, any material having a relatively low heat transfer coefficient may be used. Since heat transfer is a surface phenomena, it is also advantageous to use geometries having low surface to volume ratios, for example, spherical or cylindrical geometries. Insulators and geometry should be used to best advantage whenever temperature and/or heat flux is to be controlled.

Heating pouch housing 404 is molded to accommodate POC vial 112 and CF vial 114. Heating pouch assembly 116 may be removed from device housing 102 or it may be disposed in the housing within the built-in heating pouch holder 406. For stability, heating pouch assembly 116 may be positioned in a vertical orientation using the device housing 102 as a base as shown in FIG. 4.

In one embodiment, exothermal chemical device 402 may contain super-saturated sodium acetate trihydrate (10 g or less of 15, 20, 25, or 30% w/w water/sodium acetate mixtures). Initiating a mechanical disturbance, for example, bending a metal disk located on the exothermal chemical device initiates nucleation and an exothermic crystallization of this saturated solution and achieves a temperature of approximately 45° C. This heat will promote faster silica drying. Other exothermic chemical heating reactions may also be used, as would be apparent to one of ordinary skill in the art. Other (exothermic or endothermic) chemical reactions may not yield a constant temperature over time (i.e., a temperature plateau). Temperature regulation can be introduced into these systems using thermally activated phase-change materials (“PCMs”) (e.g., a paraffin, wax or polymer, salt hydrates, or non-paraffin organics) that melts (or freezes, boils, or condenses) at the desired temperature. In some embodiments, PCMs may be encapsulated in carbohydrate spheres. The advantage of phase-change materials is that they can be customized to very specific temperatures. Temperature is regulated at the latent heat of absorption until all the material undergoes phase change. One such example is Paraffin C21-C50 which has a melting temperature in the range 58° C.-60° C. Many different types of materials can act as PCMs, for example, metals, inorganic compounds, inorganic eutectics, and organic compounds. Exemplary PCM materials are manufactured by Rubitherm Co., such as RT64, which refers to a wax that is advertised to melt at 64° C. and RT100, which refers to a wax that is advertised to melt at 100C. Exothermic reactions for such purposes are typically activated by exposure to air humidity, oxygen, or by bringing two reaction components in close contact. Such mixtures can achieve temperatures ranging from slightly above body temperature to over 100° C.

The triggering or sudden nucleation of a supercooled solution is an exothermic reaction. One example of such a reaction, acetate crystallization, CH₃COONa₍ₗ₎→CH₃COONa₍ₛ₎, is a simple phase change reaction. For example, a flask of water, supersaturated with sodium acetate at an elevated temperature (e.g., 73.1 g per 50 ml of water at 70° C.), and then allowed to cool to room temperature (which usually takes approximately 3 hours), is relatively stable if kept pure, but if it is seeded with a small crystal of sodium acetate, activated via mechanical friction or shock (for example with a metal clicker), exposed to an electrical current, or even if dust is allowed to settle on it, it will begin to crystallize. In general, a supercooled solution can be triggered to crystallize by seeding it with the same anhydrous or hydrated crystals, mechanical friction or shock (e.g., metal clicker, metallic snap disc, sharp needles, shaking, etc.), or exposure to electrical currents. This reaction emits a considerable amount of heat (approximately 250 J/g), and when it begins to fuse, the mixture will almost instantly jump to the melting point of sodium acetate (45-55° C.). The crystallization of other supercooled substances may produce different temperatures. On the other hand, if kept sealed, the mixture is quite stable; it can be poured, moved around, etc. Since this reaction is itself a phase change reaction, the temperature remains constant without the need to add a separate phase change material, e.g., a paraffin.

The introduction of initial crystal seeds of the same solute or other similar crystalline substances, the size of the seeds, the manner in which the seeds are added, and the processing or handling of the melt after the addition of the seeds are controllable factors which are effective in precipitating nucleation. Nucleation of supercooled liquid solutions can also be induced by surface energy in the form of dislocations and surface charge on a variety of materials (seeds) when they are in an active state. PCMs can be nucleated by adding sodium tetraborate decaydrate, sodium sulfite heptahydrate, or the like. The temperature produced by the crystallization reaction can be controlled by, for example, adding another material to the supercooled liquid solution to form a mixture. For example, when ethylene glycol is added to some PCMs, the temperature produced at crystallization decreases in accordance with the amount of ethylene glycol added. Ethylene glycol is also effective to limit the size of the crystals produced when the supercooled liquid solution is triggered.

POC vial 112 is a commercially available 1.5-mL Eppendorf vial. Contained within POC vial 112 is a target-specific primer such as an HIV gag gene-specific primer. CF vial 114 is also a commercially available 1.5-mL Eppendorf vial. Contained within the CF vial 114 are generic primers for non-specific transcription of extracted nucleic acid such as random hexamers. cDNA generated from generic primers permits a more comprehensive sequence analysis since no genetic information has been lost in the reverse transcription step.

While a preferred embodiment for disposable sample processing unit 100 and device housing 102 has been described above, by way of example, one of ordinary skill in the art will appreciate that variations in structure and configuration can be made without departing from the scope of the present invention.

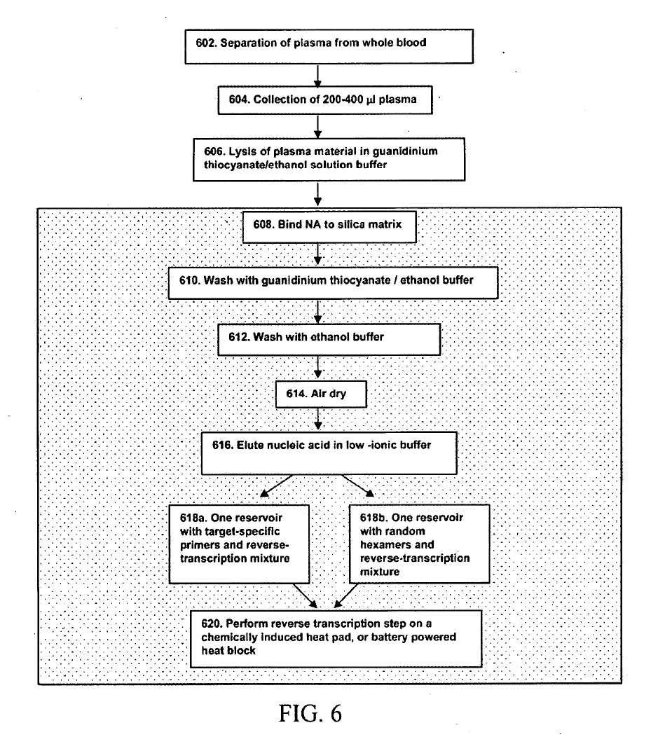

FIG. 6 is a flow chart of the process for using the disposable sample processing unit to extract and stabilize viral RNA in specimens at the point of collection. This process is merely exemplary and may include only some of the outlined steps and may include additional steps. It is noted that the steps outside the shaded box may occur outside the disposable sample processing unit 100, and steps inside the shaded box may occur within the disposable sample processing unit 100.

The process shown in FIG. 6 uses a hand-operated filter dial 302 to direct reagents and air from hand-operated syringes 106, 108, 110 through a silica matrix filter 308 via the fluidic plumbing of unit 100. In step 602, plasma is separated from whole blood. In step 604, preferably 200-400 μl of plasma are collected. In step 606, the plasma material is lysed in a guanidinium thiocyanate/ethanol solution buffer. Thereafter, in step 608, the lysed specimen is introduced via sample port 200 while filter dial 302 is in position 1, allowing nucleic acid (NA) to bind to silica matrix 308. The lysed specimen may be introduced to sample port 200 using syringe 106. Thereafter, in steps 610 and 612 silica matrix 308 is washed, first with a guanidinium thiocyanate/ethanol buffer and then with an ethanol buffer via sample port 200 while filter dial 302 is in position 1. The buffers may be introduced to sample port 200 in steps 610 and 612 through separate syringes. Filtered waste drains to the vented waste reservoir 214.

After introducing these washes, in step 614, the user repositions filter dial 302, turning it clockwise until it clicks in vertical position 2. The user then introduces air from empty syringe 108 via drying air port 204 to dry silica matrix 308. The user will then also initiate exothermal chemical device 402 by clicking a small disk on the backside of the device contained within heating pouch assembly 116. Bending the metal disk contained on exothermal chemical device 402 initiates nucleation and an exothermic crystallization of this supersaturated solution and achieves a temperature of approximately 45° C. The heat promotes faster silica drying. In another embodiment silica may be dried just through air drying.

After roughly two minutes and once drying is complete, in step 616, the user repositions filter dial 302 by turning it clockwise until it clicks to position 3. On-board eluent 228 is disposed within eluent intake channel 210 and enclosed within a frangible membrane. The user moves on-board eluent 228 onto silica matrix 308 by forcing air into the eluent intake channel 210 from empty syringe 110. The force of the air pressure bursts a frangible membrane at both ends of the eluent allowing it to flow. The user then uses the same syringe to force a second burst of air through the channel, displacing the eluent onto silica matrix 308 such that the nucleic acid is eluted in a low ionic buffer. In one embodiment, eluent exhaust channel 224 is bifurcated by eluent splitter 226 so as to allow equal portions of eluent to flow into vial ports 218. In step 618a and 618b, eluent splitter 226 evenly divides eluent into POC vial 112 and CF vial 114 by means of carefully designed channel geometry, as would be apparent to one of ordinary skill in the art, such as, for example, that used in multi-tip pipettes designed to evenly distribute volumes of fluid. POC vial 112 may contain target specific primers and a reverse-transcription mixture. CF vial 114 may contain random hexamers and a reverse-transcription mixture. Alternatively, the POC vial 112 and CF vial 114 may contain other chemicals or reagents for stabilization of purified RNA. In another embodiment, the POC vial 112 and CF vial 114 may contain no additional chemicals.

In step 620, the user then unscrews both POC vial 112 and CF vial 114 from device housing 102, caps them, and inserts them into heating pouch housing 404 on the back of device housing 102. For stability, the pouch may be positioned in a vertical orientation using the cartridge as a base as shown in FIG. 4. Because exothermal chemical device 402 has already been initiated during the silica drying process, reverse transcriptase commences automatically as the eluate warms through conduction and convection. Alternatively, POC vial 112 and CF vial 114 may be heated on a separate battery-powered heat block.

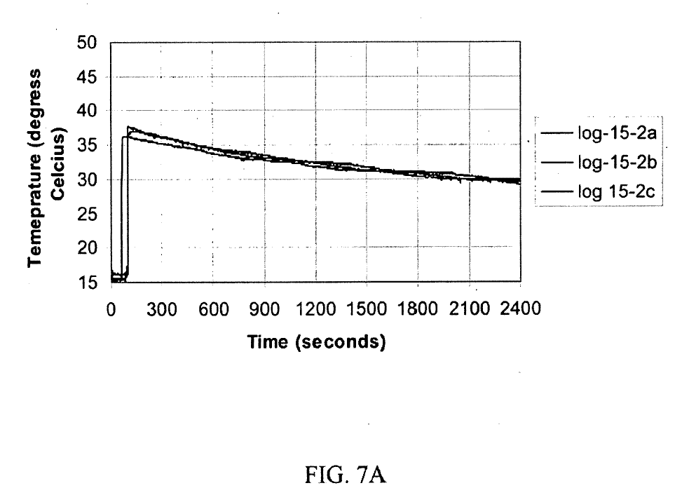

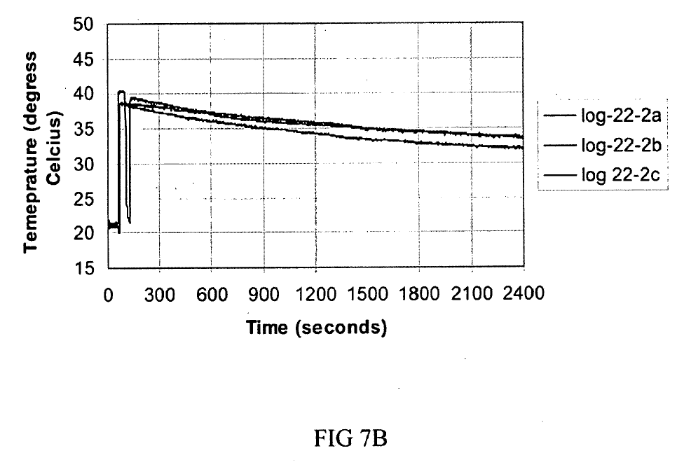

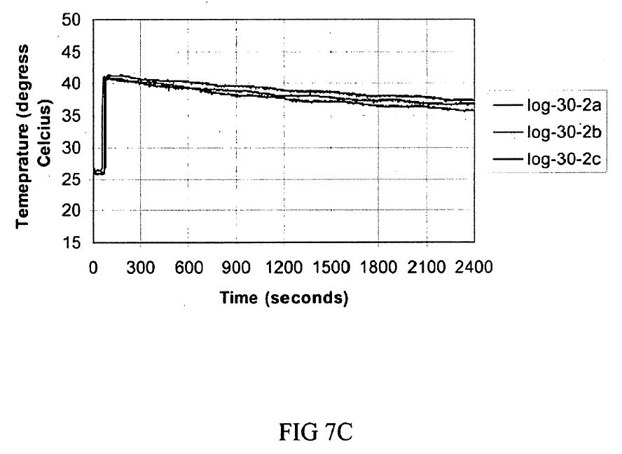

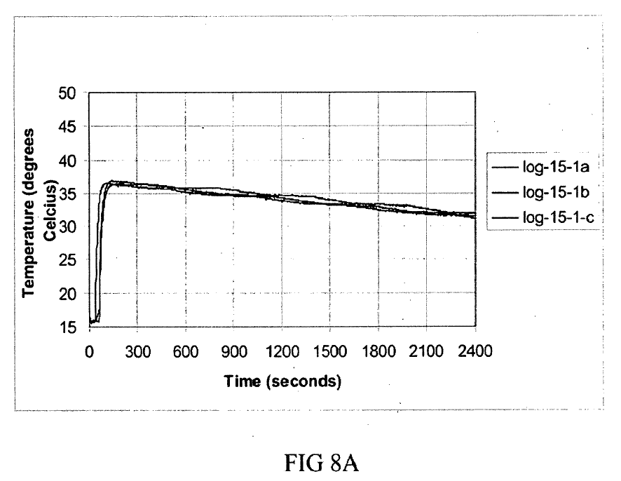

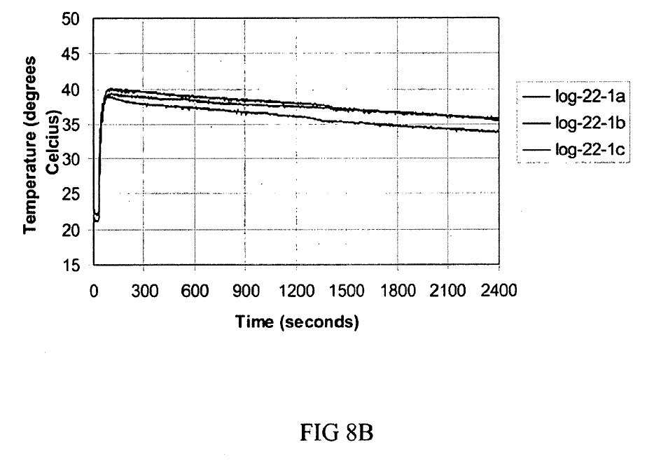

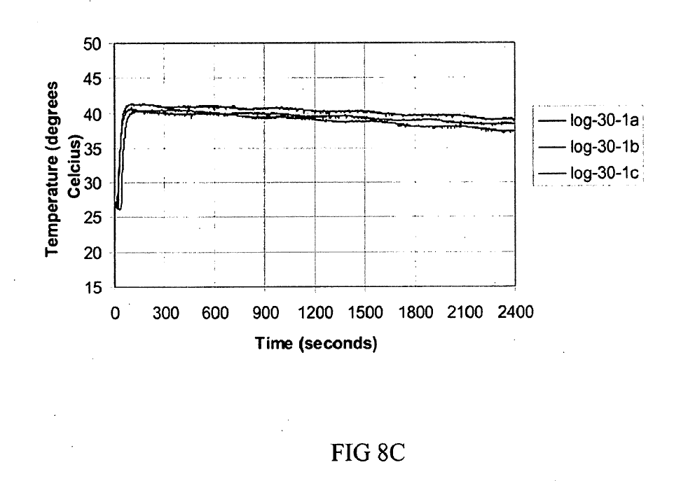

As mentioned, chemical temperature control can be used in reverse transcription (RT) at the point-of-care. In one embodiment, a mixture of sodium acetate trihydrate and water is capable of generating sufficient heat to convert RNA to cDNA over a range of ambient temperatures. To demonstrate this capability, a 25% water/sodium acetate mixture was used. An eppendorf with an RT mixture was immersed in this heat mixture. The experiments were conducted at three ambient temperatures: 15° C., 22° C., and 30° C. in triplicate. The generated heat profiles are shown for the first 40 minutes in FIGS. 7 A-C (for the heat mixture at each temperature) and FIGS. 8 A-C (for the eppendorf with an RT mixture at each temperature).

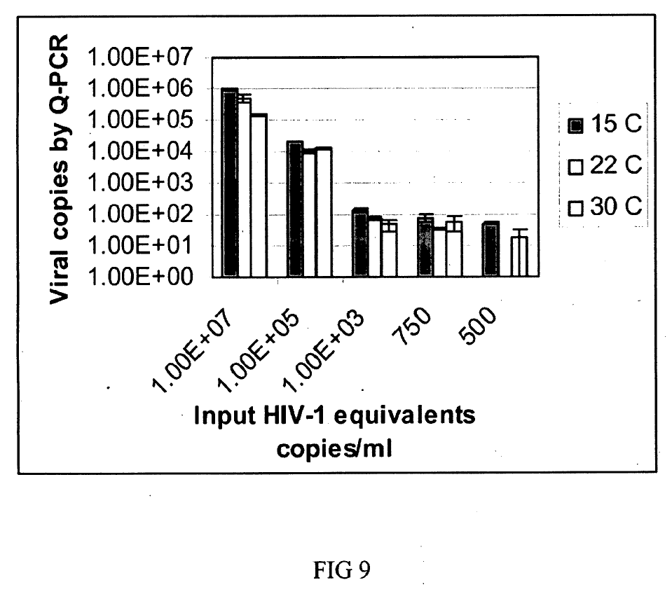

Similar heat profiles (under the same three ambient temperatures) were conducted for heat mixtures comprising 0% and 15% water/sodium acetate mixtures. The heat profiles were conducted on a PCR heat block using high to low HIV-1 template copy numbers, and the efficiency of the RT was compared to that of the Biocentric one-step RT-PCR conditions for the same viral copy number templates. This is shown in FIG. 9 as a plot of the viral copies by Q-PCR vs. input HIV-1 equivalents copies/ml. This data shows that the temperature profiles are dependent on ambient temperature, but that the RT step is tolerant to these temperature ranges. These combined data sets demonstrate that an exothermic mixture (for example sodium acetate trihydrate) can be used to provide sufficient energy to efficiently execute RT of viral pathogen RNA for diagnostics purposes at multiple ambient temperature conditions. Furthermore, this data shows that this methodology is relevant over the clinically relevant dynamic range of HIV-1 viral load 500 to 1×10⁷ copies/ml.

Current silica capture protocols for NA purification utilize vacuum pumps and or centrifuges to bind NA to a silica matrix, evaporate ethanol after the final wash, and elute the NA in small volumes of low-ionic-strength buffers. According to the present invention, a non-instrumented approach can be used to perform all these steps with commercially available components. A broad range of commercially available syringes can be used for delivery of load wash and elute RNA to the filter surface. The disposable sample processing unit of the present invention is easy-to-use, low cost, easy-to-manufacture and provides an output applicable to multiple downstream applications.

A simple device that extracts RNA at the point of specimen collection provides alternative options for stabilizing specimen RNA for shipment to testing facilities. A simple disposable device that integrates RNA extraction from clinical samples with reverse transcription to generate cDNA in a sterile container while preserving the specimen at clinical POC sites would circumvent the need to stabilize RNA in clinical specimens using cold chain. The cDNA would be stable to be posted to the appropriate clinical/surveillance laboratory for full sample characterization. Development of an inexpensive, disposable, easy-to-use device that generates cDNA from a clinical sample at the POC facility would be an invaluable tool for HIV-1 viral load testing, surveillance, molecular diagnostics, and clinical research. Genetic material in the form of cDNA can be used both immediately in a POC molecular diagnostic tool and also shipped to a central facility for detailed molecular characterization (i.e., diagnostics, cloning, sequencing, etc.).

The foregoing description of the embodiments are presented for purposes of illustration and description. The description is not intended to be exhaustive or to limit the invention to the precise form disclosed, and obviously many modifications and variations are possible in light of the above teachings. While this invention has been particularly shown and described with reference to preferred embodiments thereof, it will be understood by those skilled in the relevant art(s) that various changes in form and details may be made therein without departing form the spirit and scope of the invention. For example, the use of chemical temperature controls is not limited to assays. Thus, the breadth and scope of the present invention should not be limited by any of the above-described exemplary embodiments, but should be defined only in accordance with the following claims and their equivalents.

What is claimed is:

1. A method for processing a clinical sample at a point-of-care site comprising the steps of:
(a) collecting a clinical sample from a patient at a point-of-care site;
(b) extracting nucleic acid from said clinical sample at the point-of-care site; and
(c) preserving the extracted nucleic acid at the point-of-care site;
wherein the steps of extracting and preserving the nucleic acid occur in a sample processing unit.

2. The method of claim 1, wherein the step of preserving comprises heating the extracted nucleic acid in the sample processing unit.

3. The method of claim 1, wherein the step of extracting comprises filtering the clinical sample in the sample processing unit.

4. The method of claim 1, wherein the step of extracting comprises adding a reagent comprising chemicals for extraction of nucleic acid to the clinical sample.

5. The method of claim 1, wherein the step of preserving comprises introducing the extracted nucleic acid to a reagent comprising chemicals or biochemical agents for conversion of RNA to complimentary DNA.

6. A sample processing kit, comprising:
(a) a first syringe for holding a clinical sample in a chaotropic lysis buffer;
(b) a second syringe for holding a first wash buffer
(c) a third syringe for holding a second wash buffer
(d) a fourth syringe for holding drying air;
(e) a fifth syringe for holding air for fluidly motivating an eluent;
(f) a vial containing a reagent comprising chemicals or biochemical agents for conversion of RNA to complimentary DNA; and
(g) a disposable device comprising:
(i) ports adapted to fluidly engage said first, second, third, fourth, and fifth syringes, and said vial;
(ii) a fluidic network;
(iii) a nucleic acid capture matrix for extraction of either DNA or RNA or both from said clinical sample; and
(iv) a chemical heating element.

7. The sample processing kit of claim 6, wherein said chemical heating element comprises an exothermic phase change material that generates heat as a consequence of crystallizing a supercooled liquid and generates heat at a constant temperature as a consequence of the liquid form of the exothermic phase change material being in equilibrium with the solid form of the exothermic phase change material.

8. The sample processing kit of claim 7, wherein said exothermic phase change material is sodium acetate.

9. The sample processing kit of claim 6, wherein said chemical heating element comprises an exothermic chemical reagent mixture and a temperature regulating element comprising a phase change material that keeps the temperature generated by the exothermic chemical reagent mixture constant for a duration by being partially converted from its solid form to its liquid form.

10. The sample processing kit of claim 6, wherein the disposable device further comprises a selectively movable filter operatively connected to said fluidic network, allowing manual switching of said filter between fluid channels of said fluidic network.

11. The sample processing kit of claim 10, wherein said filter comprises a filter membrane situated inside of a fluidic channel.

12. The sample processing kit of claim 6, wherein the disposable device further comprises a frangible membrane located in the fluidic network.

13. The sample processing kit of claim 12, wherein the frangible membrane encloses said eluent.

14. A disposable device comprising:
(a) a sample port;
(b) a fluidic network;
(c) a nucleic acid capture matrix for extraction of either DNA or RNA or both from a clinical sample;
(d) a chemical heating element;
(e) a selectively movable filter operatively connected to said fluidic network, allowing manual switching of said filter from one fluid channel to another fluid channel of said fluidic network; and
(f) a collection port.

15. The disposable device of claim 14, wherein said chemical heating element comprises an exothermic phase change material that generates heat as a consequence of crystallizing a supercooled liquid and generates heat at a constant temperature as a consequence of the liquid form of the exothermic phase change material being in equilibrium with the solid form of the exothermic phase change material.

16. The disposable device of claim 15, wherein said exothermic phase change material is sodium acetate.

17. The disposable device of claim 14, wherein said chemical heating element comprises an exothermic chemical reagent mixture and a temperature regulating element comprising a phase change material that keeps the temperature generated by the exothermic chemical reagent mixture constant for a duration by being partially converted from its solid form to its liquid form.

18. The disposable device of claim 14, further comprising a first air port for drying said nucleic acid capture matrix and a second air port for receiving air adapted to motivate an eluent within the fluidic network.

19. The disposable device of claim 14, further comprising an additional collection port.

20. The disposable device of claim 14, further comprising a dial for selectively moving the filter.
